(19) United States
(12) Patent Application Publication
(10) Pub. No.: US 2026/0245676 A1
(43) Pub. Date: Aug. 20, 2026

(54) OPTIMIZATION METHODS FOR MATERIAL COMBINATION OF FACADE RETROFIT MODULE BASED ON GENETIC ALGORITHM AND PHYSARUM POLYCEPHALUM ALGORITHM

(71) Applicant: HARBIN INSTITUTE OF TECHNOLOGY, Harbin (CN)

(72) Inventors: Qi DONG, Harbin (CN); Chong GUO, Harbin (CN); Cheng SUN, Harbin (CN); Xingchi YAN, Harbin (CN); Xunzhi YIN, Harbin (CN); Dayang WANG, Harbin (CN)

(73) Assignee: HARBIN INSTITUTE OF TECHNOLOGY, Harbin (CN)

(21) Appl. No.: 19/437,338

(22) Filed: Dec. 31, 2025

(30) Foreign Application Priority Data
Feb. 14, 2025 (CN) 202510164389.1

(51) Int. Cl. G16C 60/00; G16C 20/70

(52) U.S. Cl. CPC G16C 60/00; G16C 20/70

(57) ABSTRACT

Disclosed is an optimization method for material combination of a facade retrofit module based on a genetic algorithm and a Physarum polycephalum algorithm. The method includes: dividing material categories; establishing a genetic algorithm model; based on the obtained core cavity distribution scheme, generating a path in a core cavity distribution framework using the Physarum polycephalum algorithm to form a solid material part and an other cavity part, to provide support for a core filling material and reserve space for filling an other filling material; and performing combination assembly.

### CROSS-REFERENCE TO RELATED APPLICATIONS

This application claims priority to the Chinese Patent Application No. 202510164389.1, filed on Feb. 14, 2025, the entire contents of which are incorporated herein by reference.

### TECHNICAL FIELD

The present disclosure relates to the field of building energy conservation and facade retrofit technology, and in particular, to an optimization method for material combination of a facade retrofit module based on a genetic algorithm and a Physarum polycephalum algorithm.

### BACKGROUND

With transformation of urbanization in China and advancement of quality upgrading and and stock renewal, renovation and upgrading of existing buildings have occupied an important position in building energy conservation and emission reduction. A total floor area of the existing buildings in China is about 80 billion square meters, presenting a large-scale and widely distributed demand for retrofit. Meanwhile, from energy conservation to green and low-carbon, the existing buildings in China are transitioning from a traditional “energy-saving retrofit” stage to a carbon-neutrality-oriented “green and low-carbon retrofit” stage.

Traditional external wall retrofits mostly improve thermal insulation performance by unidirectionally laying insulation boards or applying thermal insulation coatings on an outer layer of an original facade. However, these methods are often static or passive, making it difficult to perform adaptive regulation for changing climate conditions over a long time span. The resulting problems include as follows:

(1) Inability to effectively cope with seasonal and diurnal temperature differences: Under climate change and future climate warming, traditional retrofit methods lack dynamic regulation capability.

(2) Neglecting a potential of “dynamic performance materials” in facade retrofit: Most current common facade retrofit solutions only focus on thermal conductivities or thicknesses of traditional insulation materials, and fail to integrate materials with performance regulating functions, such as phase change materials (PCMs) (heat absorption and release capability) and airbags (inflatable and deflatable characteristics), into an “integrated retrofit module”. This misses an opportunity to significantly improve energy efficiency by utilizing dynamic thermal regulation.

(3) Difficulty in balancing an overheating risk and an overall structural requirement: Although some external wall systems attempt to incorporate energy storage materials or ventilation layers in a surface layer, they lack a systematic and intelligent optimal design, making it difficult to balance a plurality of objectives such as thermal insulation, heat dissipation, a structural strength, and a construction cost.

(4) Low degree of technology integration and high construction complexity: Traditional retrofit methods mostly involve on-site decentralized construction, sequentially laying or hanging a plurality of materials. The process is cumbersome and requires high technical skills from construction personnel. Once local damage occurs or material replacement is needed, large-area demolition and reinstallation are often required, resulting in high maintenance costs and a lack of flexible solutions with “replaceability” or “mobility”, i.e., interchangeability.

Therefore, it is desired to provide an optimization method for material combination of a facade retrofit module based on a genetic algorithm and a Physarum polycephalum algorithm. By deeply integrating advanced materials, including phase change materials, airbags, etc., and insulation materials with intelligent algorithms, an integrated design of an external wall retrofit module is achieved, taking into account multi-dimensional requirements such as an insulation effect, a material utilization rate, and the construction cost, and having significant promotion and application value.

### SUMMARY

One or more embodiments of the present disclosure provide an optimization method for material combination of a facade retrofit module based on a genetic algorithm and a Physarum polycephalum algorithm. The method includes dividing the facade retrofit module into a core performance component and a structure. The core performance component includes a core cavity part and a core filling material. The core filling material includes a material with performance regulating function including a phase change material and an airbag. The structure includes a solid material part, an other cavity part, and an other filling material. The solid material part refers to a structural material. The other filling material includes a thermal insulation foaming agent located in the other cavity part. The method further includes establishing a genetic algorithm model and performing multi-objective optimization on a cavity position, a cavity size, and a cavity distribution of the core cavity part with objectives of heat transfer performance optimization, core material efficiency optimization, and overall reliability optimization, to obtain an optimal or near-optimal core cavity distribution scheme. The method further includes based on the optimal or near-optimal core cavity distribution scheme, generating a path in a core cavity distribution framework using the Physarum polycephalum algorithm to form the solid material part and the other cavity part, to provide support for the core filling material and reserve space for filling the other filling material, so that the solid material part, the other cavity part, and the other filling material together form the structure. The core performance component and the structure together constitute the facade retrofit module. This enables the facade retrofit module to have thermal insulation performance, a material utilization rate, and a structural stability.

One or more embodiments of the present disclosure provide an electronic device. The electronic device includes a memory and a processor. The memory stores a computer program. The processor, when executing the computer program, implements the optimization method for material combination of a facade retrofit module based on the genetic algorithm and the Physarum polycephalum algorithm as described above.

One or more embodiments of the present disclosure provide a non-transitory computer-readable storage medium. The storage medium is configured to store computer instructions. When the computer instructions are executed by a processor, the optimization method for material combination of a facade retrofit module based on the genetic algorithm and the Physarum polycephalum algorithm as described above is implemented.

The embodiments of the present disclosure include, but are not limited to the following beneficial effects:

(1) Dynamic response to climate change and an overheating risk: By introducing variable performance components such as the phase change materials or inflatable/deflatable airbags into core cavities, an entire external wall module can actively or passively absorb heat, release heat, or adjust internal gas content according to temperature changes. This significantly alleviates overheating problems in high temperature conditions and reduces heat loss in low-temperature environments.

(2) Multi-objective optimization and efficient material utilization: Based on multi-objective optimization of the genetic algorithm, comprehensive control of a heat transfer coefficient, a total cavity volume, and a number of cavities is achieved. This avoids blindly increasing thicknesses of thermal insulation materials or using excessive solid materials, improving thermal insulation efficiency while reducing material waste.

(3) Structural integrity and construction convenience: Branching paths generated by the Physarum polycephalum algorithm provide reliable support for dynamic components such as airbags and reserve reasonable space for layouts of other thermal insulation materials. This ensures stability of the external wall retrofit module, shortens an on-site construction period, and achieves convenience for integrated installation and later maintenance.

(4) Strong adaptability and broad application prospects: The integrated retrofit module proposed in some embodiments of the present disclosure can be customized according to different climate zones, building types, or energy consumption goals. The integrated retrofit module can further integrate more new functional materials, such as photothermal conversion films, embedded renewable energy components, or the like, having broad market promotion and application potential.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering indicates the same structure, wherein:

FIG. 1 is a flowchart illustrating an exemplary process for an optimization method for material combination of a facade retrofit module based on a genetic algorithm and a Physarum polycephalum algorithm according to some embodiments of the present disclosure;

FIG. 2 is a logic diagram illustrating division of a facade retrofit module according to some embodiments of the present disclosure;

FIG. 3 is a schematic diagram illustrating a process for an optimization method for material combination of a facade retrofit module based on a genetic algorithm and a Physarum polycephalum algorithm according to some embodiments of the present disclosure;

FIG. 4 is a schematic diagram illustrating an overall effect according to some embodiments of the present disclosure;

FIG. 5 is a schematic diagram illustrating a core filling material-a core cavity part according to some embodiments of the present disclosure;

FIG. 6 is a longitudinal section view illustrating a solid material part, an other cavity part, and an other filling material according to some embodiments of the present disclosure; and

FIG. 7 is a cross-section view illustrating a solid material part-an other cavity part/an other filling material according to some embodiments of the present disclosure.

Description of reference numerals: 1, core performance component; 2, structure; 3, core cavity part; 4, core filling material; 5, solid material part; 6, other cavity part; 7, other filling material.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios in accordance with these drawings without creative labor. Unless obviously obtained from the context or the context illustrates otherwise, the same numeral in the drawings refers to the same structure or operation.

It should be understood that the terms “system,” “device,” “unit” and/or “module” used herein are a way to distinguish between different components, elements, parts, sections, or assemblies at different levels. However, the terms may be replaced by other expressions if other words accomplish the same purpose.

As shown in the present disclosure and in the claims, unless the context clearly suggests an exception, the words “one,” “a,” “an,” “one kind,” and/or “the” do not refer specifically to the singular, but may also include the plural. Generally, the terms “including” and “comprising” suggest only the inclusion of clearly identified steps and elements, however, the steps and elements that do not constitute an exclusive list, and the method or apparatus may also include other steps or elements.

Flowcharts are used in the present disclosure to illustrate the operations performed by a system according to embodiments of the present disclosure, and the related descriptions are provided to aid in a better understanding of the method and/or system. It should be appreciated that the preceding or following operations are not necessarily performed in an exact sequence. Instead, steps can be processed in reverse order or simultaneously. Also, it is possible to add other operations to these processes or to remove a step or steps from these processes.

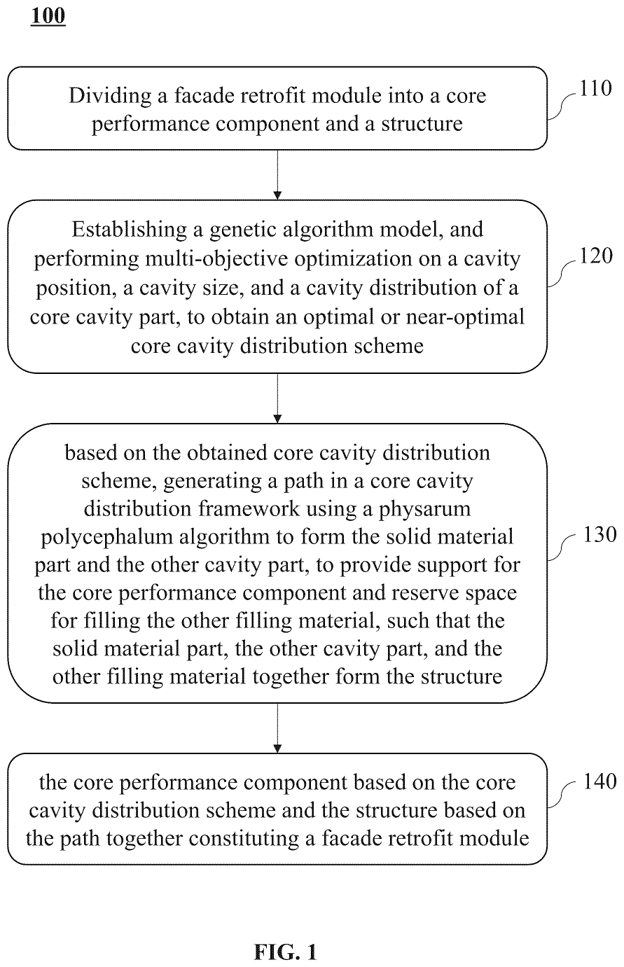

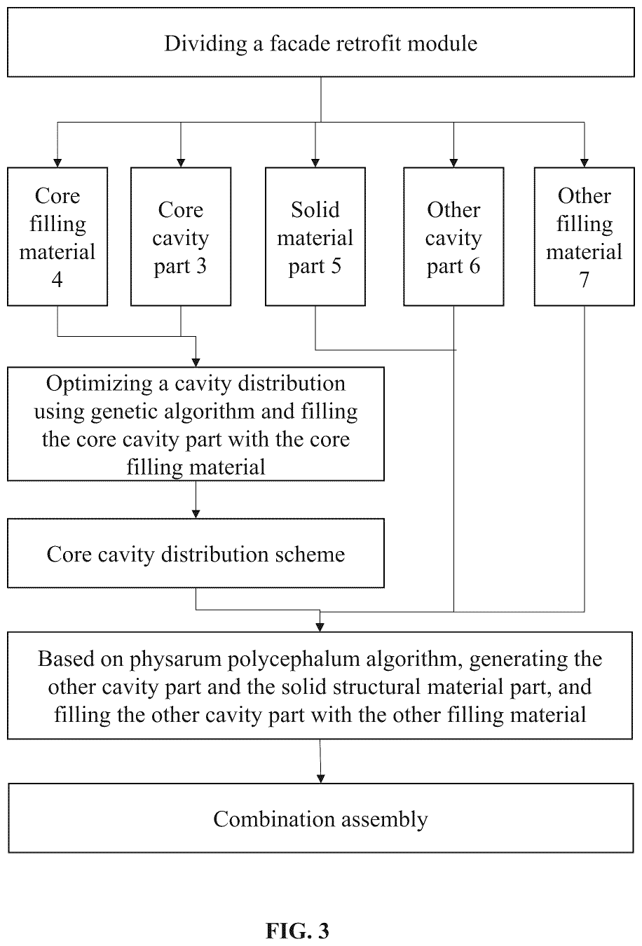

FIG. 1 is a flowchart illustrating an exemplary process for an optimization method for material combination of a facade retrofit module based on a genetic algorithm and a Physarum polycephalum algorithm according to some embodiments of the present disclosure. FIG. 3 is a schematic diagram illustrating a process for an optimization method for material combination of a facade retrofit module based on a genetic algorithm and a Physarum polycephalum algorithm according to some embodiments of the present disclosure.

As shown in FIG. 1, a process 100 includes the following steps. In some embodiments, the process 100 may be performed by a processor.

Step 110, the facade retrofit module is divided into a core performance component and a structure.

The facade retrofit module refers to a unitized component used for retrofitting, upgrading, or renovating an existing building facade.

The core performance component refers to a core functional part in the facade retrofit module that is responsible for providing main performance (e.g., thermal performance, light regulation performance, ventilation performance, etc.).

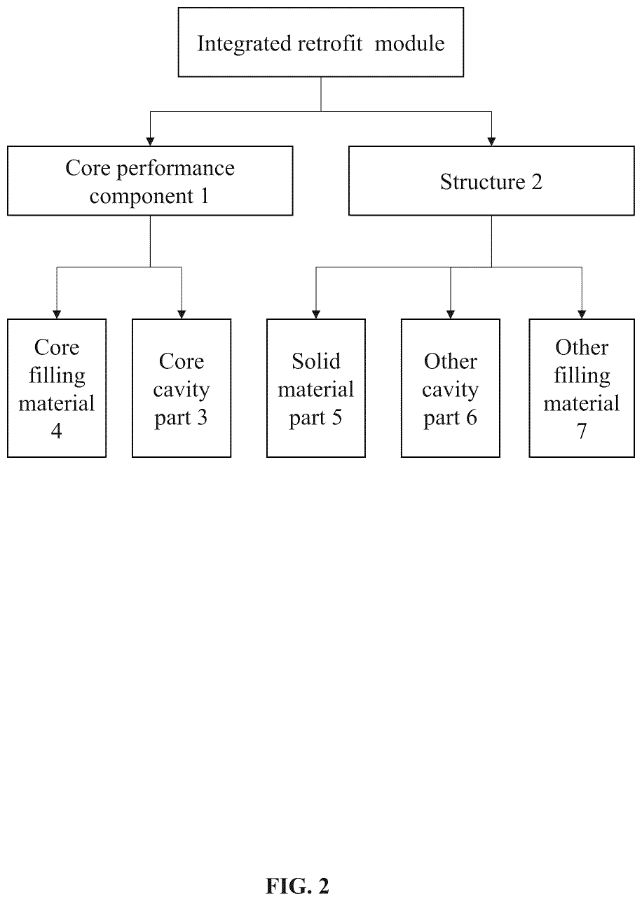

FIG. 2 is a logic diagram illustrating division of a facade retrofit module according to some embodiments of the present disclosure.

In some embodiments, as shown in FIG. 2, the core performance component 1 includes a core cavity part 3 and a core filling material 4.

The core cavity part 3 refers to a space inside the core performance component 1 for accommodating a filling material or forming an air gap to achieve specific performance. The core cavity part 3 may include one or more core cavities.

In some embodiments, the core cavity part 3 may be designed as a multi-layer micro-cavity structure. A space of the core cavity part 3 is divided into a plurality of small air chambers by partitions to utilize low thermal conductivity of air to provide excellent basic thermal insulation performance. In some embodiments, the core cavity part 3 is an internal space designed to accommodate the core filling material 4. A geometric shape and a volume of the core cavity part 3 need to precisely match a morphology and an amount of selected filling material. For example, if the core filling material 4 includes a plate-shaped phase change material, a shape of the core cavity part 3 should be designed as a flat cavity adapted thereto, and a volume of the core cavity part 3 needs to precisely match a total size of the required phase change material plate to ensure tight filling and avoid gaps. As another example, if the core filling material 4 includes an inflatable airbag, the volume of the core cavity part 3 should be designed to accommodate a maximum volume of the airbag after inflation, while the geometric shape of the core cavity part 3 should match an external contour of the airbag to achieve efficient performance regulation and avoid jamming.

In some embodiments, the core cavity part 3 may be implemented through various structural forms, including but not limited to a single large cavity, a multi-layer micro-cavity structure, or a cavity with a complex three-dimensional geometric shape.

The core filling material 4 refers to a material combination filled in the core cavity part 3 to provide main performance regulation functions for the core performance component 1. In some embodiments, the core filling material 4 includes, but is not limited to a material with performance regulating function including a phase change material, an airbag, etc. Components with the performance regulating function are all applicable to the method of the embodiments of the present disclosure. For example, the core filling material 4 may include an intelligent film material, etc.

The phase change material refers to a material that absorbs or releases a large amount of latent heat by undergoing a physical phase change (e.g., solid-liquid phase change) within a specific temperature range, thereby regulating a temperature of a surrounding environment. The phase change material may include, but is not limited to, organic or inorganic phase change materials such as paraffin, fatty acid esters, salt hydrates, etc.

The airbag refers to a flexible container or bag for containing gas or a variable medium, and serves as a component of a material with the performance regulating function in the module.

In some embodiments, the airbag in the core filling material 4 may be an inflatable flexible airbag. A thickness of the airbag may be dynamically adjusted by inflation or deflation.

The structure 2 refers to an external or supporting part of the retrofit module that provides support, load-bearing, enclosure, and other auxiliary functions.

In some embodiments, as shown in FIG. 2, the structure 2 includes a solid material part 5, an other cavity part 6, and an other filling material 7.

The solid material part 5 refers to a main frame and enclosure constituting the structure 2, e.g., a solid material that provides mechanical strength and support functions.

In some embodiments, the solid material part 5 refers to a structural material. A structural material refers to a material capable of withstanding external loads and maintaining a geometric shape of the module. For example, the structural material includes common structural materials such as wood, concrete, or the like.

The other cavity part 6 refer to a space inside the facade retrofit module other than the core cavity part 3, which is used to accommodate filling materials or form auxiliary air gaps.

The other filling material 7 refer to a material filled in the other cavity part 6 to provide auxiliary functions such as thermal insulation, sound insulation, or fire protection. In some embodiments, the other filling materials 7 include a thermal insulation foaming agent located in the other cavity part 6.

The thermal insulation foaming agent refers to a chemical agent or prefabricated material that forms a foam material with a large number of closed-cell structures by foaming and expansion and is used to fill cavities to provide excellent thermal resistance performance.

In some embodiments, the solid material part 5 may serve as a frame and internal support grid of the module, providing structural stability and durability. In some embodiments, the structural material may adopt various engineering materials, including but not limited to metals (e.g., steel, aluminum), wood, concrete, engineering plastics, and various new high-performance composite materials.

In some embodiments, the other cavity part 6 may serve as an auxiliary thermal insulation layer, e.g., a closed air cavity located at a periphery of the module, to further reduce heat loss. For example, the other cavity parts 6 may also be used to accommodate electrical wires, sensor lines, or small control components, serving a purpose of concealment and protection. In some embodiments, the arrangement of the other cavity part 6 may be flexible and diverse, including but not limited to a single cavity, separated multiple cavities, or channels reserved for specific functions.

In some embodiments, the thermal insulation foaming agent in the other filling material 7 may include but is not limited to polyurethane, phenolic, polystyrene, rock wool, glass wool, etc.

In some embodiments, the division of the facade retrofit module may be achieved through conceptual zoning in a design phase. For example, in a preliminary design of the module, a designer may divide the entire module on a drawing into a “core” region (i.e., the core performance component 1) that bears primary thermal performance functions and a “structural” region (i.e., the structure 2) that provides mechanical support and peripheral protection, based on functional requirements.

In some embodiments, the division of the facade retrofit module may also be achieved through physical prefabrication and assembly. For example, the core performance component 1 is produced as an independent prefabricated unit and then integrated and assembled on-site or in a factory with the structure 2 that serves as an external frame. In some embodiments, the division of the facade retrofit module may also be performed in various other ways. For example, the division may be based on functional zoning, material property classification, or considerations of lifecycle stages.

Step 120, a genetic algorithm model is establish, and multi-objective optimization is performed on a cavity position, a cavity size, and a cavity distribution of the core cavity part, to obtain an optimal or near-optimal core cavity distribution scheme.

In some embodiments, objectives of the multi-objective optimization on the cavity position, the cavity size, and the cavity distribution of the core cavity part 3 are “heat transfer performance optimization, core material efficiency optimization, and overall reliability optimization.”

The genetic algorithm model refers to a computational model that simulates natural selection and genetic mechanisms. The genetic algorithm is an optimization algorithm that searches for an optimal solution or a near-optimal solution in a solution space through iterative operations of “selection, crossover, and mutation.”

The heat transfer performance optimization refers to a process of adjusting cavity parameters (e.g., the cavity position, the cavity size, and the cavity distribution) of the core cavity part 3 to minimize heat loss or gain of the core cavity part 3 under specific environmental conditions, thereby achieving objectives of energy saving and comfort improvement.

The core material efficiency optimization refers to a process of minimizing a usage amount or cost of the core filling material 4 (e.g., the phase change material, the airbag) or maximizing a performance-to-cost ratio of the core filling material 4 while meeting performance requirements.

The overall reliability optimization refers to a process of evaluating and ensuring structural integrity, performance stability, and expected functions of various components of the facade retrofit module not to fail due to material aging, environmental factors, or changes in operating conditions during long-term operation.

The cavity position refers to a three-dimensional spatial coordinate or a relative position of a core cavity of the core cavity part 3 inside the facade retrofit module. The cavity size refers to a geometric size parameter of the core cavity of the core cavity part 3. The cavity distribution refers to arrangement, density, and geometric configuration of core cavities in the facade retrofit module.

In some embodiments, the cavity size, the cavity position, and the cavity distribution are respectively determined by a center point coordinate, a shape, and a size parameter of the core cavity. In some embodiments, the cavity position may be a three-dimensional coordinate (x, y, z) of a core cavity in a module region, and the cavity size may be a size parameter r of the core cavity. The three-dimensional coordinate (x, y, z) of the core cavity in the module region is the three-dimensional coordinate of a center point of the core cavity in the module.

For example, for a spherical core cavity, the cavity position may be determined by a sphere center coordinate (i.e., the center point coordinate) (x, y, z). A shape of the spherical core cavity is spherical, and a size parameter is a radius r. The cavity size may be determined based on a geometric method (e.g., a geometric method for calculating a spherical volume based on the radius). For a cubic core cavity, the cavity position may be determined by a geometric center coordinate (i.e., the center point coordinate). A shape of the cubic core cavity is cubic, and a size parameter may be a side length. The cavity size may be determined based on a geometric method (e.g., a geometric method for calculating a cubic volume based on the side length). The processor may determine the cavity positions and the cavity sizes of all core cavities, thereby determining an overall cavity distribution.

In some embodiments, a cavity volume of the core cavity part 3 is a total cavity volume of all core cavities of the core cavity part 3, and a number of cavities is a total number a of core cavities whose size parameter is not 0. For example, if the core cavity part includes a plurality of independent core cavities, the total cavity volume is obtained by simply summing volumes of all core cavities. In some embodiments, a calculation method for the total cavity volume may also be determined based on an actual geometric shape and a distribution of the cavities. For example, the calculation may be performed based on an integration method or a geometric summation method. In design parameterization, if a size parameter (e.g., a radius) of a core cavity is set to 0, the core cavity is considered to be non-existent and is not counted in the total count. Only cavities that actually exist and have physical dimensions are counted.

In some embodiments, for the heat transfer performance optimization, in a rapid iteration stage, a simplified thermal resistance network may be used to evaluate relative heat transfer performance to determine a heat transfer coefficient. This allows for faster comparison of advantages and disadvantages of different design schemes during a algorithm convergence process. The simplified thermal resistance network simplifies material layers and cavities within the facade retrofit module into thermal resistance elements connected in series or in parallel. This enables rapid calculation of an approximate heat transfer coefficient of the module, avoiding time-consuming detailed simulations in each iteration. For a final optimized or candidate scheme, a more comprehensive three-dimensional heat transfer analysis should be performed using numerical simulation methods such as finite element simulation to obtain a more accurate heat transfer coefficient.

In some embodiments of the present disclosure, by refining cavity parameters into the center point coordinate, the shape, and the size, and combining with a simplified thermal resistance network for rapid iterative evaluation, a precise description of complex cavity geometry and acceleration of the optimization process are achieved. This enables rapid comparison of a plurality of cavity distribution schemes while ensuring computational efficiency, significantly improving design efficiency and optimization accuracy.

The multi-objective optimization refers to an optimization process that simultaneously considers two or more conflicting optimization objectives and searches for a set of solutions that perform well across all objectives. For example, the multi-objective optimization refers to optimization that takes the heat transfer performance optimization, the core material efficiency optimization, and the overall reliability optimization as objectives.

In some embodiments, multi-objective optimization may be performed using a variety of algorithms and strategies, including but not limited to Pareto dominance-based evolutionary algorithms, indicator-based evolutionary algorithms, or hybrid optimization combining other heuristic algorithms (e.g., particle swarm optimization, simulated annealing).

In some embodiments, an objective of the heat transfer performance optimization is using the simplified thermal resistance network to evaluate the relative heat transfer performance in the rapid iteration stage, which includes dividing the facade retrofit module into regular network units, dividing the facade retrofit module into n layers along a heat transfer direction, and each layer having m units, with series connection between the layers and parallel connection within a layer.

In some embodiments, for the heat transfer performance optimization, the processor may determine an overall total heat transfer coefficient based on a total number of layers, a number of units in each layer, and a thermal resistance of each unit. For example, a minimum value of the overall total heat transfer coefficient is determined according to the following formula (1) and formula (2):

$${f}_{1} = minimize ( {U}_{total} = \frac{1}{{R}_{total}} ) , \qquad ( 1 )$$

$${R}_{total} = {\sum }_{k = 1}^{n} \frac{1}{{\sum }_{j = 1}^{m} \frac{1}{{R}_{k , j}}} , \qquad ( 2 )$$

where Uₜₒₜₐₗ denotes the overall total heat transfer coefficient, Rₜₒₜₐₗ denotes an overall total thermal resistance, f₁ denotes the objective of the heat transfer performance optimization, n denotes the total number of layers of the module along a heat flow direction (typically perpendicular to the plane of the external wall); m denotes the number of units in each layer; and R_{k,j} denotes a thermal resistance of a j-th unit in a k-th layer. The formula (1) aims to minimize the overall total heat transfer coefficient, i.e., maximize the overall total thermal resistance, to achieve better thermal insulation performance.

In some embodiments, a thermal resistance of each unit may be determined by the following formula (3):

$${R}_{k , j} = \frac{{d}_{k , j}}{{λ}_{k , j} \cdot {A}_{k , j}} , \qquad ( 3 )$$

where d_{k,j} denotes a thickness (a leng of a heat transfer path) of the j-th unit in the k-th layer; λ_{k,j} denotes a thermal conductivity of the j-th unit in the k-th layer; and A_{k,j} denotes a cross-sectional area of the j-th unit in the k-th layer.

In some embodiments, a process for the heat transfer performance optimization may also be iteratively executed according to an iteration process of the genetic algorithm model.

The core material efficiency optimization means less core cavity volume. In some embodiments, for the core material efficiency optimization, the processor may determine a minimum value of the cavity volume of the core cavity part 3 based on volumes of one or more core cavities. For example, the minimum value of the cavity volume is determined according to the following formula (4):

$${f}_{2} = minimize ( {\sum }_{i = 1}^{a} {V}_{i} ) , \qquad ( 4 )$$

where Vᵢ denotes a volume of an i-th cavity, and f₂ denotes an objective of the core material efficiency optimization. The formula (4) aims to optimize the total cavity volume of all core cavities. For example, by reducing the cavity volume, a filling amount of the phase change material or the airbag material may be directly reduced while meeting heat transfer performance requirements, thereby reducing a material cost and improving a material utilization efficiency.

In some embodiments, a process for the core material efficiency optimization may also be iteratively executed according to the iterative process of the genetic algorithm model.

The overall reliability optimization is to minimize the number of cavities as much as possible to avoid structural complexity and an excessively high failure rate. In some embodiments, for the overall reliability optimization, the processor may determine a minimum value of the number of cavities a. For example, the minimum value of the number of cavities a is determined according to the following formula (5):

$${f}_{3} = minimize ( a ) , \qquad ( 5 )$$

where f₃ denotes an objective of the overall reliability optimization. The formula (5) aims to minimize the number of cavities a as much as possible. A smaller number of cavities may simplify a manufacturing and assembly process of the module, reduce risks of air leakage or heat leakage caused by issues such as material connection and poor sealing, thereby improving long-term reliability of the module.

In some embodiments, a process for overall reliability optimization may also be iteratively executed according to the iterative process of the genetic algorithm model.

In some embodiments, a fitness function of the genetic algorithm model comprehensively considers the heat transfer coefficient, the cavity volume, and the number of cavities in a weighted method, and weights of various items may be dynamically adjusted according to actual requirements.

In some embodiments, by comprehensively balancing the three optimization objectives described above, the processor may perform weighted summation on f₁, f₂, and f₃ to determine an optimal or near-optimal core cavity distribution scheme. For example, a weighted summation formula is the following formula (6):

$$F ( x ) = {\overline{ω}}_{1} {f}_{1} + {\overline{ω}}_{2} {f}_{2} + {\overline{ω}}_{3} {f}_{3} , \qquad ( 6 )$$

where F(x) represents a score of a corresponding scheme; ω̅₁, ω̅₂, and ω̅₃ are weight coefficients of the three objectives f₁, f₂, and f₃, respectively; and ω̅₁+ω̅₂+ω̅₃=1.

For example, if a designer focuses more on energy-saving performance, a weight ω̅₁ of the objective of the heat transfer performance optimization f₁ may be increased. If the designer focuses more on cost control, a weight ω̅₂ of the objective of the core material efficiency optimization f₂ may be increased. The weight coefficients of different objectives may be dynamically adjusted according to actual requirements to adapt to optimization priorities of different projects or different stages.

In some embodiments, in addition to the weighted summation method, the multi-objective optimization may also adopt other methods, e.g., a Pareto dominance-based method, goal programming method, or multi-attribute decision analysis method.

In some embodiments of the present disclosure, by precisely defining quantitative optimization objectives for the heat transfer performance optimization, the core material efficiency optimization, and the overall reliability optimization, and combining weighted summation, refined multi-objective optimization of a core cavity distribution of the facade retrofit module is achieved. This includes efficiently evaluating heat transfer performance using a simplified thermal resistance network, optimizing the core cavity volume to reduce material cost, and reducing the number of cavities to improve structural reliability. This comprehensive optimization method can find the optimal balance among performance, cost, and reliability.

In some embodiments, an iteration termination condition of the genetic algorithm model includes a fitness function showing no significant improvement within a preset number of iterations, or obtaining an optimal solution set meeting a preset convergence criterion.

The fitness function showing no significant improvement within the preset number of iterations refers to that after the genetic algorithm model runs for a certain number of iterations, a variation amplitude of the fitness function of the model is lower than a preset improvement threshold. The improvement threshold may be preset based on experience.

The obtaining the optimal solution set meeting the preset convergence criterion refers to that iteration is terminated early when the algorithm finds a solution that meets specific performance requirements or achieves predetermined optimization objectives. In some embodiments, the obtaining the optimal solution set meeting the preset convergence criterion may also be determined by user manual intervention to stop the process, or based on a preset upper limit of computing resource consumption, e.g., CPU time.

In some embodiments of the present disclosure, by clarifying the iteration termination condition of the genetic algorithm, computing efficiency and optimization accuracy are effectively balanced. For example, by setting that the fitness function does not improve significantly or reaches a preset convergence criterion as the iteration termination condition, unnecessary long-time computing is avoided. This ensures that high-quality optimization results are obtained within a reasonable time, reduces computing cost, and improves efficiency of a design process.

Step 130, based on the obtained core cavity distribution scheme, a path in a core cavity distribution framework is generated using a Physarum polycephalum algorithm to form the solid material part and the other cavity part, to provide support for the core performance component and reserve space for filling the other filling material, such that the solid material part, the other cavity part, and the other filling material together form the structure.

The Physarum polycephalum algorithm refers to a bio-inspired optimization algorithm that simulates growth and branching behaviors of a Physarum polycephalum in nature. By simulating a search and connection process of the Physarum polycephalum to nutrient sources or target points, the Physarum polycephalum algorithm can efficiently generate a network structure with high connection efficiency, short paths, or low material consumption.

The core cavity distribution framework refers to a spatial region or boundary condition that includes the core cavity distribution scheme, serving as an operation domain for the Physarum polycephalum algorithm to generate a support path.

The path refers to geometric representations of a series of connection points, lines, or networks generated in the core cavity distribution framework by the Physarum polycephalum algorithm. These geometric representations may define boundaries and shapes of the solid material part and the other cavity part.

In some embodiments, the processor may use a Physarealm plugin of Grasshopper to generate the path in the core cavity distribution framework based on the Physarum polycephalum algorithm. The plugin may simulate behaviors of the Physarum polycephalum. By placing attraction points (e.g., connection points or force-bearing points of the module) and repulsion points (e.g., the core cavity part itself) within the core cavity distribution framework, the algorithm generates an efficient path network connecting these points according to a set growth rule, e.g., a diffusion speed, an attraction intensity, or a path decay rate.

In some embodiments, the Physarum polycephalum algorithm limits growth and branching morphology by setting a growth rule, a boundary condition, and an objective function for the support path, to form a supporting skeleton meeting an installation requirement of the core performance component within a cavity distribution range, while minimizing occupancy of structural solid material and preserving space fillable by a thermal insulation material.

In some embodiments, the growth rule may include simulating a response of the Physarum polycephalum to a nutrient concentration gradient, e.g., a diffusion speed, an attraction intensity, and a path decay rate. For example, when a path encounters a “nutrient source,” a growth speed of the path accelerates and the path branches toward the source. When the path moves away from the source, a conductivity of the path decays. In some embodiments, the boundary condition may define operational space limitations of the algorithm. For example, the boundary condition may limit that a path is not capable of penetrating an interior of the core cavity part, or must connect to a specific structural anchor point of the module. In some embodiments, the objective function may be set to minimize a total length of the generated path to reduce material consumption, or to minimize a stress of the generated skeleton structure to improve structural strength. For example, the objective function may combine structural mechanics analysis to ensure that the generated path can effectively bear a weight of the core performance component. In some embodiments, the growth rule, the boundary condition, and the objective function may also be set in various ways, including but not limited to based on empirical parameters, based on physical simulation feedback, or adaptive adjustment through other optimization algorithms.

In some embodiments, the Physarum polycephalum algorithm includes constructing nodes and edges, setting source/sink points, iteratively executing the Physarum polycephalum algorithm, and outputting an optimal path.

In some embodiments, constructing the nodes and the edges includes: regarding key positions in an optimized cavity distribution as a node set V; allowing potential connections between nodes (within a certain range) to form an edge set E; letting G=(V, E) representing a graph, where V is the node set (the cavity distribution optimized by the genetic algorithm), and E is the edge set available for connection (potential connections between nodes); and for each edge eᵢⱼ∈E, defining a pipe thickness as Dᵢⱼ.

The key positions in the optimized cavity distribution may be core cavities, connection points, etc. The node set V is a set of the key positions. The key positions are a basis for the growth path of the Physarum polycephalum algorithm. For example, geometric center points of the optimized core cavity part, or connection points between the core cavity part and a boundary of the structure may be used as the node set V.

The edge set E refers to a set of potential connections allowed to be generated between various nodes in the node set V, representing all possible connections that can be used by the Physarum polycephalum algorithm to construct paths. For example, a maximum connection distance threshold may be set. A potential edge eᵢⱼ is established between two nodes to form the edge set E only when a Euclidean distance between the two nodes is less than the maximum connection distance threshold.

The graph G refers to an abstract network structure composed of the node set V and the edge set E, represented as G=(V, E). The graph G is a basis for optimization search by the Physarum polycephalum algorithm.

The pipe thickness Dᵢⱼ refers to a parameter assigned to each edge eᵢⱼ in the graph G, representing a “conductivity” or “material thickness” of the edge, which is used to measure a capability of the edge in flow transmission. For example, a pipe thickness Dᵢⱼ may be initialized to a small positive value (e.g., 0.1 or 1), which ensures that all potential edges have a certain “conductivity” at the beginning of the algorithm, allowing for the initial flow in the network. As another example, an initial value of the pipe thickness Dᵢⱼ may also be set based on a distance between nodes or importance of the nodes in the module. For instance, edges connecting key areas may be assigned a larger initial thickness.

In some embodiments, setting source/sink points includes: in a module structure, regarding a core cavity or a support area as a “source” node, and regarding an external area or another functional area as a “sink” node; and defining a plurality of source-sink pairs according to a material layout to be supported.

The source/sink point refers to a specific node in the Physarum polycephalum algorithm that simulates supply and consumption of nutrients or resources, thereby guiding a growth direction of a path. For example, a geometric center point of a core cavity or several points on a surface of the core cavity may be set as the “source” nodes because the core performance component 1 needs to obtain structural support from the “source” nodes. Simultaneously, external connection points of the module (e.g., anchor points connected to a main building structure) or areas requiring heat conduction may be set as the “sink” nodes, representing endpoints that a structural path needs to extend to reach.

In some embodiments, a plurality of source-sink pairs may be defined according to a material layout to be supported. For example, if there are a plurality of independent phase change material areas inside the module that need to obtain support separately, each phase change material area may be defined as a separate “source”, and a corresponding area in the structure 2 that needs to provide connection support may serve as a “sink” corresponding to the “source”.

In some embodiments, iteratively executing the Physarum polycephalum algorithm includes: initializing a thickness

$${D}_{ij}^{( 0 )}$$

of all edges to a positive value; and updating a flow rate fᵢⱼ according to a pressure difference.

In some embodiments, the processor may update the flow rate fᵢⱼ according to formulas (7) and (8):

$${\sum }_{j ∈ ( j )} {f}_{ij} = \{ \begin{matrix}- {I}_{0} , if j = inlet \\ {I}_{0} , if j = outlet \\ 0 , other nodes\end{matrix} , \qquad ( 7 )$$

$${f}_{ij} = \frac{{D}_{ij}}{{L}_{ij}} \cdot ( {p}_{i} - {p}_{j} ) , \qquad ( 8 )$$

where fᵢⱼ denotes a flow rate of an edge, pᵢ and pⱼ denote a pressures of a node i and a pressure of a node j, respectively, and Lᵢⱼ denotes a length. A linear equation system for pᵢ is obtained according to the formula (7) and the formula (8), to obtain the pressures pᵢ of all nodes and the flow rates of respective edges. The formula (7) defines a flow balance for each node, where I₀ represents an flow rate magnitude of inflow or outflow, indicating that a flow rate I₀ flows in at a source node, a flow rate I₀ flows out at a sink node, and the flow rates at other nodes are conserved. The formula (8) describes a relationship between a flow rate and a pipe thickness Dᵢⱼ, a length Lᵢⱼ, and the pressures pᵢ and pⱼ at two end nodes, i.e., the flow rate is proportional to the thickness and a pressure difference, and inversely proportional to the length. For example, in each iteration, the flow rate is first calculated according to a current pipe thickness and a length, and then node pressures are derived inversely according to a flow balance equation.

In some embodiments, the processor may further update a thickness Dᵢⱼ of an edge according to a pipe evolution equation:

$$\frac{{dD}_{ij}}{dt} = μ ( \vert {f}_{ij} \vert - λ {D}_{ij} ) , \qquad ( 9 )$$

where Dᵢⱼ denotes a conductivity or thickness of the edge eᵢⱼ, representing a flow capacity; μ denotes a forward gain coefficient, controlling a speed of pipe growth; λ denotes a decay rate, indicating that a pipe gradually atrophies when not used; and |fᵢⱼ| denotes an absolute flow rate, where a larger flow rate makes a pipe healthier and thicker. If the flow rate is small or even zero, the pipe thickness continuously decays until disappearance. Because in this simulation process, the growth path needs to pursue a shortest path and material saving on one hand, and needs to consider minimum structural support and safety redundancy on the other hand, the following formula (10) is applied on the path after final updating is completed:

$${D}_{ij}^{( final )} = \{ \begin{matrix}{D}_{ij}^{( iter end )} , if \vert {D}_{ij}^{( iter end )} \vert \geq δ \\ δ , if ε \leq \vert {D}_{ij}^{( iter end )} \vert \leq δ \\ 0 , if \vert {D}_{ij}^{( iter end )} \vert > ε\end{matrix} , \qquad ( 10 )$$

where Δt denotes a step size, δ denotes a pipe thickness threshold, and ε denotes a flow rate threshold for determining whether to retain a pipe. The formula (10) performs discretization and trimming on the pipe thickness, aiming to remove fine and unimportant connections and standardize retained paths. For example, if a final thickness

$${D}_{ij}^{( iter end )}$$

of an edge is greater than or equal to δ, an original thickness of the edge is retained. If the final thickness is between ε and δ, the thickness is uniformly set to δ to ensure a certain structural strength. If the final thickness is less than ε, the pipe is considered to be unimportant, and the final thickness is set to 0, indicating that the path is removed.

In some embodiments, outputting an optimal path includes: after iterating a plurality of steps, thicknesses of most pipes with small flow rates gradually decreasing and disappearing; retaining pipes with large flow rates, to form an optimal or near-optimal skeleton path capable of supporting the core performance component 1.

The optimal path refers to a network structure retained after iterative optimization by the Physarum polycephalum algorithm, which has a large flow rate, strong conductivity, and may efficiently connect the source/sink nodes. The optimal path forms a skeleton capable of supporting the core performance component 1.

In some embodiments, after iterating a number of steps, the thicknesses of most pipes with small flow rates gradually decrease and disappear. When Dᵢⱼ decreases below a preset flow rate threshold ε, the edge is marked as 0 in a final path post-processing, thereby being removed from the network, simulating pruning of inefficient or redundant connections by an organism.

In some embodiments, edges that continuously obtain a larger flow rate during an iteration process, and whose Dᵢⱼ continuously increases in the pipe evolution equation are ultimately retained. These retained pipes represent paths with the highest transmission efficiency between source/sink points, forming the supporting skeleton for the core performance component 1. For example, in a path post-processing stage, only edges whose final thickness is greater than or equal to a preset pipe thickness threshold δ are retained and serve as components of the skeleton path.

In some embodiments, after the algorithm runs to reach a preset number of iterations or a convergence condition, all edges with Dᵢⱼ greater than ε and connected nodes of the all edges may be extracted and output as three-dimensional geometric data (e.g., line segments, curves, or thin tubes). The three-dimensional geometric data may be imported into relevant software for further refined design and manufacturing.

In some embodiments of the present disclosure, by constructing the nodes and the edges, setting source/sink points, iteratively performing the Physarum polycephalum algorithm, and outputting the optimal path, a structural supporting skeleton of the facade retrofit module can be efficiently generated. By simulating a biological growth process, a complex network structure with low material consumption and high connection efficiency is generated.

Step 140, the core performance component based on the core cavity distribution scheme and the structure based on the path together constitute the facade retrofit module, such that the facade retrofit module as a whole possesses thermal insulation performance, a material utilization rate, and a structural stability.

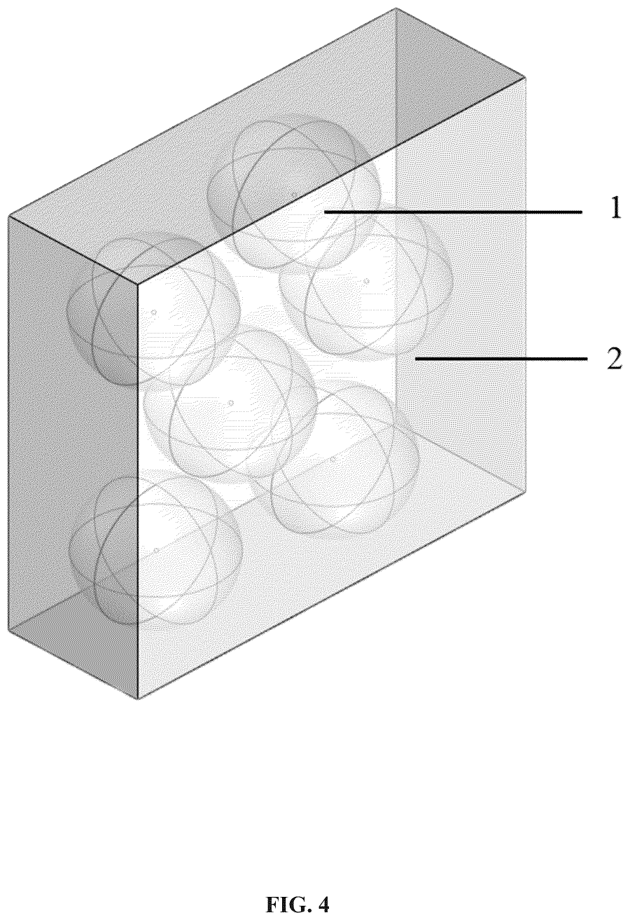

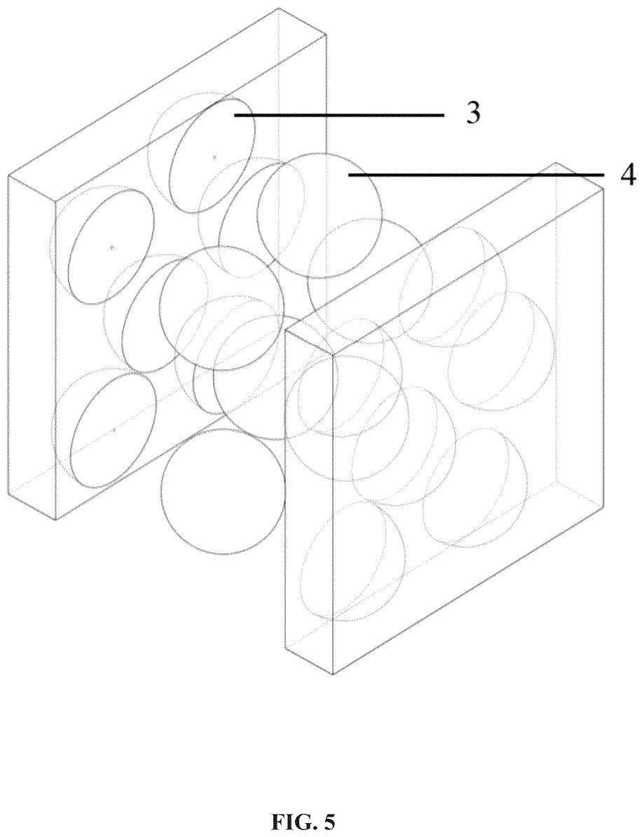

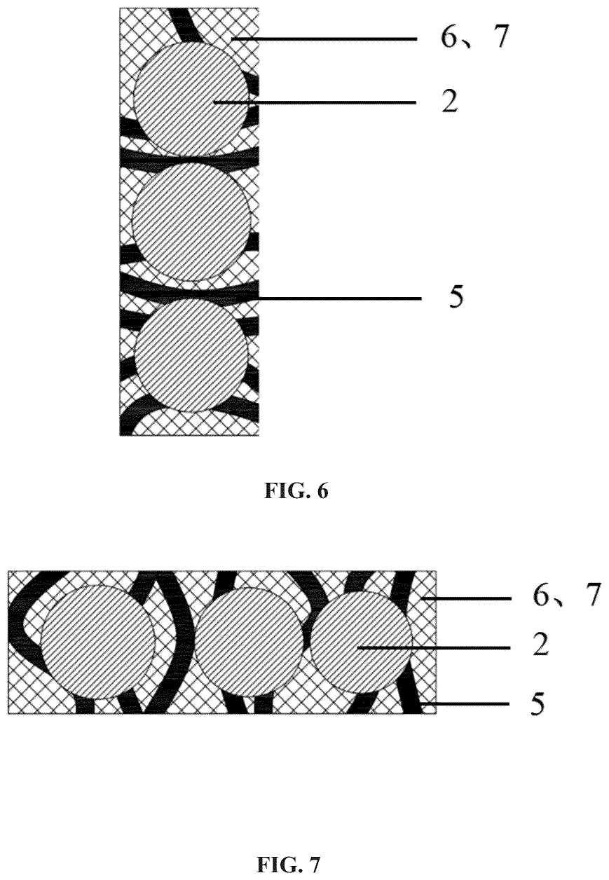

FIG. 4 is a schematic diagram illustrating an overall effect according to some embodiments of the present disclosure. FIG. 5 is a schematic diagram illustrating a core filling material-a core cavity part according to some embodiments of the present disclosure. FIG. 6 is a longitudinal section view illustrating a solid material part, an other cavity part, and an other filling material according to some embodiments of the present disclosure. FIG. 7 is a cross-section view illustrating a solid material part-an other cavity part/an other filling material according to some embodiments of the present disclosure.

In some embodiments, the structure 2 may be constructed based on the path generated in step 130, the core performance component 1 may be constructed based on the core cavity distribution scheme in step 120, and the core performance component 1 and the structure 2 may be assembled and combined, to form a complete facade retrofit module. Schematic diagrams of respective parts and an overall effect diagram are shown in FIGS. 4-7.

In some embodiments, in a manufacturing process, the core performance component 1 is produced as an independent prefabricated unit, then embedded into a pre-built framework of the structure 2, and fixed and sealed, ultimately constituting the facade retrofit module. In some embodiments, a combination manner of the core performance component 1 and the structure 2 may further include, but is not limited to, integral compression molding, on-site assembly, or rapid connection using modular interfaces, etc.

Through multi-objective optimization and the phase change material and a performance regulating airbag in the core performance component 1, the facade module can efficiently regulate heat, achieving excellent thermal insulation performance, thereby reducing building energy consumption. Simultaneously, the structure 2 optimized by the Physarum polycephalum algorithm maximizes reduction of occupancy by solid material while providing necessary support, improving the material utilization rate. In addition, the optimized structure 2 ensures the structural stability of the entire retrofit module when bearing external loads.

In some embodiments, the other filling material 7 and the solid material part 5 after injection or foaming form an integral structure with the core cavity part 3, to reduce the heat transfer coefficient and improve a module strength.

For example, during a module assembly process, when the thermal insulation foaming agent (as the other filling material 7) foams and expands in the other cavity part 6, the thermal insulation foaming agent not only fills a reserved space but also closely contacts and bonds with a surrounding solid material part 5 and an external surface of the core cavity part 3, thereby forming a continuous and seamless integrated structure. As another example, if a prefabricated other filling material 7 such as a thermal insulation board is used, the board may be mechanically fixed or glued to surfaces of the solid material part 5 and the core cavity part 3 during installation, ultimately also forming a tight integral structure. In some embodiments, the formation of the integral structure may also be achieved in a plurality of ways, including, but not limited to, using heat fusion connection, welding, riveting, or using a composite material with self-adhesive properties.

In some embodiments, when the other filling material 7, the solid material part 5, and the core cavity part 3 form a tight integrated structure, gaps or voids at material interfaces can be effectively eliminated. The gaps or voids would otherwise form thermal bridges or air infiltration paths, leading to heat loss. By eliminating the thermal defects, an overall thermal resistance of the module is increased, and the heat transfer coefficient (U-value) is further reduced.

In some embodiments, when different components are tightly connected into a whole, stress can be effectively transferred and dispersed, avoiding local stress concentration, thereby enhancing compressive, bending, and shear resistance of the entire facade retrofit module. As another example, the integral structure can enhance a capability of the module to resist external loads such as wind load and seismic force, improving its durability and long-term stability.

In some embodiments of the present disclosure, the other filling material and the solid material part after injection or foaming form the integral structure with the core cavity part, which eliminates thermal bridges and gaps, effectively reducing the heat transfer coefficient. Simultaneously, an overall structural strength and rigidity of the module are enhanced, improving a capability to resist external loads, and significantly improving an energy-saving effect and a service reliability level of the facade retrofit module.

In some embodiments, when the facade retrofit module is entirely installed on an external wall facade of a building, the facade retrofit module may be connected to an original wall through a prefabricated component, an embedded component, or an adhesive layer, to form an integrated external wall thermal insulation retrofit system.

In some embodiments, a back side of the facade retrofit module may be fixedly connected through T-shaped or L-shaped metal hangers (prefabricated components) pre-installed on the original wall. As another example, expansion bolts may be used to anchor embedded components (e.g., pre-embedded metal plates or plastic anchors) on the module into the original wall, achieving a firm connection. In other embodiments, a high-performance building adhesive may be applied between the module and the original wall to form the adhesive layer, achieving a comprehensive and continuous connection. In some embodiments, the connection manner may also include, but is not limited to, self-tapping screws, welding, a rail-type hanging system, or a composite connection combining a plurality of connection manners.

In some embodiments of the present disclosure, by connecting the facade retrofit module to the original wall using the prefabricated component, the embedded component, or the adhesive layer to form the integrated external wall thermal insulation retrofit system, gaps or cavities that may exist between a traditional external insulation layer and a main structure are eliminated, thereby effectively preventing thermal bridge effects and air infiltration. This integrated system enables the retrofit module to not only function as an independent thermal insulation unit but also to work synergistically with the original wall, forming a continuous and efficient thermal insulation barrier.

In some embodiments of the present disclosure, through a layered and decoupled module design, combined with the multi-objective optimization of the core cavity using the genetic algorithm and generation of an efficient support structure using the Physarum polycephalum algorithm, heat transfer performance, material efficiency, and structural reliability are effectively balanced. This significantly improves energy-saving effect of a building, reduces retrofit cost, and enhances a service life and environmental adaptability of the module.

It should be noted that the above description of the process 100 is for the purpose of exemplification and illustration only and does not limit the scope of application of the present disclosure. For those skilled in the art, various corrections and changes can be made to the process 100 under the guidance of the present disclosure. However, these corrections and changes remain within the scope of the present disclosure.

In some embodiments of the present disclosure, an electronic device including a memory and a processor is also provided. The memory stores a computer program. The processor, when executing the computer program, implements the optimization method for material combination of a facade retrofit module based on the genetic algorithm and the Physarum polycephalum algorithm according to any embodiment of the present disclosure.

In some embodiments of the present disclosure, a non-transitory computer-readable storage medium for storing computer instructions is also provided. When the computer instructions are executed by a processor, the optimization method for material combination of a facade retrofit module based on the genetic algorithm and the Physarum polycephalum algorithm according to any embodiment of the present disclosure is implemented.

The memory in some embodiments of the present disclosure may be a volatile memory or a non-volatile memory, or may include both volatile and non-volatile memory. The non-volatile memory may be a read only memory (ROM), a programmable read only memory (PROM), an erasable programmable read only memory (EPROM), an electrically erasable programmable read only memory (EEPROM), or a flash memory. The volatile memory may be a random access memory (RAM), which serves as an external high-speed cache. Exemplarily, many forms of RAM are available, such as a static random access memory (SRAM), a dynamic random access memory (DRAM), a synchronous dynamic random access memory (SDRAM), a double data rate synchronous dynamic random access memory (DDR SDRAM), an enhanced synchronous dynamic random access memory (ESDRAM), a synchlink dynamic random access memory (SLDRAM), and a direct rambus random access memory (DR RAM). It should be noted that the memory for the method described in the present disclosure is intended to include, but is not limited to, these and any other suitable types of memory.

In the above embodiments, implementation may be achieved entirely or partially through software, hardware, firmware, or any combination thereof. When implemented using software, the above embodiments may be implemented entirely or partially in the form of a computer program product. The computer program product includes one or more computer instructions. When the computer instructions are loaded and executed on a computer, the processes or functions described in embodiments of the present disclosure are generated entirely or partially. The computer may be a general-purpose computer, a dedicated computer, a computer network, or other programmable devices. The computer instructions may be stored in a computer-readable storage medium or transmitted from one computer-readable storage medium to another computer-readable storage medium. For example, the computer instructions may be transmitted from a website, computer, server, or data center to another website, computer, server, or data center via wired (e.g., coaxial cable, optical fiber, digital subscriber line (DSL)) or wireless (e.g., infrared, wireless, microwave, etc.) means. The computer-readable storage medium may be any available medium that may be accessed by a computer or a data storage device, such as a server or data center, that integrates one or more available media. The available medium may be a magnetic medium (e.g., floppy disk, hard disk, magnetic tape), an optical medium (e.g., digital video disc (DVD)), or a semiconductor medium (e.g., solid state disc (SSD)).

During implementation, steps of the above method may be completed by an integrated logic circuit of hardware in a processor or by instructions in a software form. The steps of the method disclosed in the embodiments of the present disclosure may be directly performed by a hardware processor, or performed by a combination of hardware and software modules in the processor. The software module may be located in a mature storage medium in the field, such as random access memory, flash memory, read only memory, programmable read only memory, electrically erasable programmable memory, or registers. The storage medium is located in the memory. The processor reads information in the memory and completes the steps of the above method in combination with its hardware. To avoid repetition, details are not described herein again.

It should be noted that the processor in the embodiments of the present disclosure may be an integrated circuit chip with signal processing capability. During implementation, steps of the above method may be completed by an integrated logic circuit of hardware in the processor or by instructions in a software form. The processor may be a general-purpose processor, a digital signal processor (DSP), an application-specific integrated circuit (ASIC), a field programmable gate array (FPGA) or other programmable logic devices, discrete gate or transistor logic devices, or discrete hardware components. The processor may implement or execute the methods, steps, and logic block diagrams disclosed in the embodiments of the present disclosure. The general-purpose processor may be a microprocessor, or the processor may also be any conventional processor. The steps of the method disclosed in the embodiments of the present disclosure may be directly performed by a hardware decoding processor, or performed by a combination of hardware and software modules in the decoding processor. The software module may be located in a mature storage medium in the field, such as random access memory, flash memory, read only memory, programmable read only memory, electrically erasable programmable memory, or registers. The storage medium is located in the memory. The processor reads information in the memory and completes the steps of the above method in combination with its hardware.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. Furthermore, the particular features, structures, or features may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for description purpose and that the appended claims are not limited to the disclosed embodiments, but, on the contrary, are intended to cover modifications and equivalent arrangements that are within the spirit and scope of the disclosed embodiments. For example, although the implementation of various parts described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%. Correspondingly, in some embodiments, the numerical parameters used in the description and claims are approximate values, and the approximate values may be changed according to the required features of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

1. An optimization method for material combination of a facade retrofit module based on a genetic algorithm and a Physarum polycephalum algorithm, comprising:
dividing the facade retrofit module into a core performance component and a structure; wherein the core performance component comprises a core cavity part and a core filling material, and the core filling material comprises a material with performance regulating function including a phase change material and an airbag; and the structure comprises a solid material part, an other cavity part, and an other filling material; wherein the solid material part refers to a structural material, and the other filling material comprises a thermal insulation foaming agent located in the other cavity part;
establishing a genetic algorithm model, and performing multi-objective optimization on a cavity position, a cavity size, and a cavity distribution of the core cavity part with objectives of heat transfer performance optimization, core material efficiency optimization, and overall reliability optimization, to obtain an optimal or near-optimal core cavity distribution scheme; the core performance component comprising the core cavity part and the core filling material; and
based on the optimal or near-optimal core cavity distribution scheme, generating a path in a core cavity distribution framework using a Physarum polycephalum algorithm to form the solid material part and the other cavity part, to provide support for the core filling material and reserve space for filling the other filling material, such that the solid material part, the other cavity part, and the other filling material together form the structure;
wherein the core performance component and the structure together constitute the facade retrofit module, such that the facade retrofit module as a whole possesses thermal insulation performance, a material utilization rate, and a structural stability.

2. The method according to claim 1, wherein the cavity size, the cavity position, and the cavity distribution are respectively determined by a center point coordinate, a shape, and a size parameter; a total cavity volume of the core cavity part is a total volume of all cavities of the core cavity part, and a number of cavities is a total number a of core cavities whose size parameter is not zero; for the heat transfer performance optimization, in a rapid iteration stage, a simplified thermal resistance network is used to evaluate relative heat transfer performance to determine a heat transfer coefficient; wherein the cavity position is a three-dimensional coordinate (x, y, z) of a core cavity in a module region; and the cavity size is a size parameter r of the core cavity.

3. The method according to claim 2, wherein an objective of the heat transfer performance optimization is using the simplified thermal resistance network to evaluate the relative heat transfer performance in the rapid iteration stage, comprising dividing the facade retrofit module into regular network units, dividing the facade retrofit module into n layers along a heat transfer direction, and each layer having m units, with series connection between the layers and parallel connection within a layer; wherein
the heat transfer performance optimization is expressed by:
$${f}_{1} = minimize ( {U}_{tota1} = \frac{1}{{R}_{total}} ) , \qquad ( 1 )$$
$${R}_{total} = {\sum }_{k = 1}^{n} \frac{1}{{\sum }_{j = 1}^{m} \frac{1}{{R}_{k , j}}} , \qquad ( 2 )$$
where Uₜₒₜₐₗ denotes an overall total heat transfer coefficient, Rₜₒₜₐₗ denotes an overall total thermal resistance, f₁ denotes the objective of the heat transfer performance optimization, n denotes a total number of layers of the facade retrofit module along a heat flow direction; m denotes a number of units in each layer; and R_{k,j} denotes a thermal resistance of a j-th unit in a k-th layer;
$${R}_{k , j} = \frac{{d}_{k , j}}{{λ}_{k , j} \cdot {A}_{k , j}} , \qquad ( 3 )$$
where d_{k,j} denotes a thickness of the j-th unit in the k-th layer; λ_{k,j} denotes a thermal conductivity of the j-th unit in the k-th layer; and A_{k,j} denotes a cross-sectional area of the j-th unit in the k-th layer;
the core material efficiency optimization is expressed by:
$${f}_{2} = minimize ( {\sum }_{i = 1}^{a} {V}_{i} ) , \qquad ( 4 )$$
where Vᵢ denotes a volume of an i-th cavity, and f₂ denotes an objective of the core material efficiency optimization;
the overall reliability optimization is expressed by:
$${f}_{3} = minimize ( a ) , \qquad ( 5 )$$
where f₃ denotes an objective of the overall reliability optimization;
a weighted summation formula is expressed by:
$$F ( x ) = {\overline{ω}}_{1} {f}_{1} + {\overline{ω}}_{2} {f}_{2} + {\overline{ω}}_{3} {f}_{3} , \qquad ( 6 )$$
where ω̅₁+ω̅₂+ω̅₃=1.

4. The method according to claim 1, wherein an iteration termination condition of the genetic algorithm model comprises a fitness function showing no significant improvement within a preset number of iterations or obtaining an optimal solution set meeting a preset convergence criterion.

5. The method according to claim 1, wherein the Physarum polycephalum algorithm limits growth and branching morphology by setting a growth rule, a boundary condition, and an objective function for a support path, to form a supporting skeleton meeting an installation requirement of the core performance component within a cavity distribution range, while minimizing occupancy of structural solid material and preserving space fillable by a thermal insulation material.

6. The method according to claim 5, wherein the Physarum polycephalum algorithm comprises:
constructing nodes and edges: regarding key positions in an optimized cavity distribution as a node set V; allowing potential connections between nodes to form an edge set E; and letting G=(V, E), representing a graph, where V is the node set, E is the edge set available for connection, and for each edge eᵢⱼ∈E, defining a pipe thickness as Dᵢⱼ;
setting source/sink points: in a module structure, regarding a core cavity or a support area as a source node, and regarding an external area or another functional area as a sink node; and defining a plurality of source-sink pairs according to a material layout to be supported;
iteratively executing the Physarum polycephalum algorithm: initializing a thickness
$${D}_{ij}^{( 0 )}$$
of all edges to a positive value; and updating a flow rate fᵢⱼ according to pressure difference:
$${\sum }_{j ∈ ( j )} {f}_{ij} = \{ \begin{matrix}- {I}_{0} , if j = inlet \\ {I}_{0} , if j = outlet \\ 0 , other nodes\end{matrix} , \qquad ( 7 )$$
$${f}_{ij} = \frac{{D}_{ij}}{{L}_{ij}} \cdot ( {p}_{i} - {p}_{j} ) , \qquad ( 8 )$$
where fᵢⱼ denotes a flow rate of an edge, and pᵢ and pⱼ denote a pressure of a node i and a pressure of a node j, respectively, and Lᵢⱼ denotes a length; wherein a linear equation system for pᵢ is obtained through the above formulas to obtain pressures pᵢ of all nodes and flow rates of edges; then Dᵢⱼ is updated according to a pipe evolution equation expressed by:
$$\frac{{dD}_{ij}}{dt} = μ ( \vert {f}_{ij} \vert - λ {D}_{ij} ) , \qquad ( 9 )$$
where Dᵢⱼ denotes a conductivity or thickness of eᵢⱼ, representing a flow capacity; μ denotes a forward gain coefficient controlling a growth speed of a pipe; λ denotes a decay rate indicating that a pipe gradually atrophies when not used; |fᵢⱼ| denotes an absolute flow rate, wherein a larger flow rate makes a pipe healthier and thicker; if the flow rate is small or even zero, the pipe thickness continuously decays until disappearance; wherein after final updating is completed, following operation is performed on a path:
$${D}_{ij}^{( final )} = \{ \begin{matrix}{D}_{ij}^{( iter end )} , if \vert {D}_{ij}^{( iter end )} \vert \geq δ \\ δ , if ε \leq \vert {D}_{ij}^{( iter end )} \vert \leq δ \\ 0 , if \vert {D}_{ij}^{( iter end )} \vert > ε\end{matrix} , \qquad ( 10 )$$
where Δt denotes a step size, δ denotes a pipe thickness threshold, and ε denotes a flow rate threshold for determining whether to retain a pipe; and
outputting an optimal path: after iterating a number of steps, thicknesses of most pipes with small flow rates gradually decreasing and disappearing; retaining pipes with large flow rates, to form an optimal or near-optimal skeleton path capable of supporting the core performance component.

7. The method according to claim 1, wherein the other filling material and the solid material part after injection or foaming form an integral structure with the core cavity part to reduce a heat transfer coefficient and improve a module strength.

8. The method according to claim 1, wherein when the facade retrofit module is entirely installed on an external wall facade of a building, the facade retrofit module is connected to an original wall through a prefabricated component, an embedded component, or an adhesive layer to form an integrated external wall thermal insulation retrofit system.

9. An electronic device, comprising a memory and a processor, the memory storing a computer program, wherein the processor, when executing the computer program, implements the optimization method for material combination of the facade retrofit module based on the genetic algorithm and the Physarum polycephalum algorithm according to claim 1.

10. A non-transitory computer-readable storage medium for storing computer instructions, wherein when the computer instructions are executed by a processor, the optimization method for material combination of the facade retrofit module based on the genetic algorithm and the Physarum polycephalum algorithm according to claim 1 is implemented.
